(19) United States
(12) Patent Application Publication
(10) Pub. No.: US 2004/0197336 A1
(43) Pub. Date: Oct. 7, 2004

(54) Bispecific antibodies in which the binding capability is reversibly inhibited by a photocleavable moiety

(76) Inventor: Colin Henry Self, Northumberland (GB)

Correspondence Address: GREENBERG TRAURIG, LLP, 885 3RD AVENUE, NEW YORK, NY, 10022, US

(21) Appl. No.: 10/829,404

(22) Filed: Apr. 20, 2004

Related U.S. Application Data
(63) Continuation of application No. 08/945,868, filed on Nov. 3, 1997, now abandoned.
Application No. 08/945,868 is a 371 of international application No. PCT/GB96/01066, filed on May 2, 1996.

(30) Foreign Application Priority Data
May 3, 1995 (GB) 9509004.9
Aug. 4, 1995 (GB) 9516606.2

(51) Int. Cl. A61K 39/395; C07K 16/46

(52) U.S. Cl. 424/155.1; 530/388.8; 530/391.1

(57) ABSTRACT

Antibodies are described which can bind to a member of a binding pair and to a macromolecule in which the capability of binding to the macromolecule is reversibly inhibited by the presence of a photocleavable moiety. Favoured antibodies are bispecific antibodies, for example against a tumour cell and against an enzyme or a killer cell in which the capability of binding to the enzyme or the killer cell is inhibited until the photocleavable moiety is removed on irradiation.

This invention relates to antibodies which are reversibly inhibited, their preparation and use. More specifically this invention relates to antibodies capable of binding to a member of a binding pair and to a macromolecule, the binding to the macromolecule being reversibly inhibited, to their preparation to their use.

It was been shown that certain proteins can be covalently bound by light labile moieties so that the proteins are prevented from expressing their biological activity until the light labile moieties are cleaved by irradiation (see for example S Thompson et ad, Biochemical and Biophysical Research Communications, 201, 1213-1219 (1994) and S Thompson et all, Biochemical Society Transactions, 255S, 23 (1995).

However, there has been no demonstration either (a) that antobody fragments may be reversibly inhibited or (b) that antibodies able to bind to more than one ligand can be reversibly inhibited with respect to only one activity. It has now been found that not only can these excepted things be done but they have a useful and surprising part to play in therapy diagnostics.

The present invention provides an antibody which is capable of binding to (a) a member of a binding pair and to (b) a macromolecule; in which the capability of binding to the macromolecule is reversibly inhibited by the presence of a photocleavable moiety.

The antibodies of this invention may be polyclonally or monoclonally derived.

The term “member of a binding pair” means a member of a pair of entities which will bind to each other when brought into contact. The skilled reader will appreciate that a common and particularly useful binding pair is an antibody and antigen pair. However, many other receptors are known to which ligands bind. Thus the antibody of this invention may be labelled with a receptor or, more commonly, labelled with a ligand that will bind to a receptor, for example it may be labelled with hormone that will bind to a hormone receptor. A list of receptors and ligands is given hereinafter.

The antibody may bind to a member of a binding pair because it is an antibody against that member of a binding pair or because it is labelled with the other member of the binding pair. Thus for example an antibody labelled with oestradiol may bind to oeseradiol receptors on a cell.

The most suitable members of a binding pair envisaged are cell bound receptors, such as tumour markers.

In a preferred class, the antibody of this invention has two active sites. This may be achieved in any convenient manner but it is presently considered most appropriate to joint two antibodies raised against two different epitopes. This type of antibody is often referred to herein as bispecific antibody. Although normally this type of antibody has two specifities, more than two is also envisaged although less preferred.

In a preferred aspect this invention provides a bispecific antibody comprising a first antibody component capable of binding a receptor and a second-antibody component capable of binding a macromolecule; said second antibody component being reversibly inhibited by the presence of a photoclevable moiety.

The first antibody component may be the whole immunoglobulin (eg IgG) or more suitably may be a part thereof which retains the active site but is free of the Fc region, for example the Fab or Fab¹₂.

The second antibody component may be the whole immunoglobin (eg IgG) or more suitably may be a part thereof which retains the active site but is free of the Fc region, for example the Fab or Fab¹₂.

Most suitably both the first and second antibody components are both parts of antibodies which retain the active sites but are free of the Fc regions, for example the Fab or Fab¹₂.

The first antibody component is directed against a target, for example it is an antibody against a tumour cell marker.

The second antibody component is directed against a macromolecule that is desired to be located on the target, for example it may be an enzyme.

Since in one aspect this invention provides a method of treating cancer by converting a pro-drug of a cytotoxic drug into the drug in the environs of a tumour, most suitable the antibody (and especially the second antibody component) is an antibody against an enzyme capable of producing a cytotoxic agent from a less cytotoxic agent. The enzyme and pro-drug are advantageously those known to be suitable for use in ADEPT (antibody directed enzyme pro-drug therapy) (see for example New Antibody Technology and the Emergence of Useful Cancer Therapy, Ed. Richard Begent and Anne Hamblin, pub Royal Society of Medicine Press, especially pages 75-77.

Such enzymes are well known to the skilled reader and include:

1. Phosphatase (particularly alkaline phosphatase) which can be used to convert less cytotoxic phosphoryrated pro-drugs into the drugs, for example etoposide phosphate, mitomycin phosphate and doxorubicin phosphate may be dephosphorylated to produce etoposide, mitomycin and doxrubicin.

2. Carboxypeptidase (particularly G2) which is able to remove glutamic acid residues from pro-drugs in which a glutamic acid residue is used to inactivate the drug.

3. β-Glucosidase which can be used to produce cyanide from amygdalin.

4. β-Lactamases (particularly penicillinase and cephalosporinase) can be used to produce viublastine or DAVLBHYD by hydrolysing the β-lactan ring of a pro-drug in which the drug is linked to cephalosporin.

5. Amidase such as a penicillin amidase such as phenoxymethyl penicillin amidase which can produce melphalan or doxorubicin from their acetamide, for example phenoxyacetamide, derivatives.

6. Cytosine deaminase which can convert s-fluorocytisine to 5-fluorouracil.

7. Nitroreductase (particularly from E Coli) which converts CB 1954 to an active aklylating agent.

It will be appreciated that by use of this invention enzymes having one or more than one binding site may be employed as desired.

The art is replete with references to target cells and the markers they contain against which antibodies may be raised. Since such cells and markers and the method of raising antibodies are commonly known the form no part of this invention as such. However, it is worthy of mention that carcino embryonic antigen found in human colorectal cancer has had monoclonal antibodies such as YPC2/12.1 raised against them my immunisation with a 108 KD glycoprotein by standard procedures. Also antibodies have been raised against the lincocyte common antigen CD45 found in lymphomas and against cytokeratins expressed by carcinoma cells and found for example in draining lymph nodes of patients with carcinoma of the breast.

Other cell tumour markers include (i) viruses: CBV and HPV; (ii) mutated or altered genes: ras, p53 connexin 37, frame shift mutations (FAP); (iii) normally silent genes: MAGE (melanoma): iv unpredulated, PEM (MUCI product); different antigens: tyrosinases, Melan Mart 1, gp 100, c-erb B2. CEA, HER2, EGFR, CA125, antibody from B cell lymphomas, T-cell receptors.

The following are also of note: (i) with respect to anti CEA for, for example, Colorectal cancers see J Natl Cancer Inst 81, 688-696 (1989) and Cancer Immun Immunother 25, 10-15 (1987), Cancer Res 45, 5769-80 (1985), Cancer 63, 1343-52 (1989); (ii) Anti-C14(LeY), Ten Feizi Biosci Rep 3, 163-170 (1993); (iii) Anti 791T/36 (TAG 72 gp),—J Cancer 43, 6153-60 (1989),Cancer Res 46, 5524-5528 (1986), Cancer Res 49, 6153-60 (1989) (Xoma); (iv) Anti CEA (CEM 231) J Immunol 141, 1053, 4060 (1988); (v) Anti-TAG 72 (CC49), Cancer Res 48, 4583-96 (1988), 50, 1291-98 (1990), 51,2965-72, 46, 2325-38 (1986); (iv) Anti KS1/4, Cancer Res 50, 3540-44 (1990), 44,681-87, Cancer Immun Immunther 28, 171-8; (vii)MAbB3, Cancer Res 51, 3781-3787; (viii) C242, J. Clin. Inves. 90,405-11 (1992); (iv) P-Glycoprotein, PNAS 83, 7785-89 (1986), Cancer Res 48, 1926-9 (1988); and (x) the antibodies referred to against inter alia colorectal, gastric and ovarian cancers referred to in J. National Cancer Institute 81, 688-696 (1989).

Thus it will be realised that a particularly preferred form of this invention comprises a bispecific antibody comprising a first antibody component capable of binding to a tumour cell and a second antibody component capable of binding ant enzyme; said second antibody component being reversibly inhibited by a photocleavable moiety.

The term “photocleavable moiety” means any agent attached to the antibody which can be removed on exposure to electromagnetic energy such as light energy of any desired variety whether visible, UV, X-ray or the like (e.g. microwave).

Suitable photocleavable moieties are well known from the art, for example Biological Applications of Photochemical Switches, Ed H Morrison, Biiorganic Photochemistry Series, Vlumne 2, J. Wiley & Sons which is incorporated herein by cross-reference, especially Chapter 1, Section 4, pages 34-50.

The photocleavable moiety employed is largely a matter of choice but it has been found to be particularly easy to employ a reagent which couples to hydroxy or amino residues present in the antibody. Thus phosgene, diphosgene, DCCl or the like may be used to generate photocleavable esters, amides, carbonates and the like from a wide range of alcohols. Generally substituted by arylalkanols are employed, particularly nitorphenyl methyl alcohol 1-nitrophenylethan-1-ol and substituted analogues. The methods of the publications of S Thompson et al referred to hereinbefore may be employed. Generally the cloaking of the antibody will take place in aqueous solution at ambient temperature. Co-solvents such as dioxane may be employed if desired. The chemistry will be familiar to the skilled worker who derivatises macromolecules such as antibodies. The references referred to in the preceding incorporated publication may be consulted if desired.

In the treatment of tumours using a pro-drug, the antibody of the invention is administered to the subject and irradiated, the enzyme (if non endogenous enzyme is to be employed) is administered to the subject and the pro-drug is administ (which is converted to the more cytotoxic drug in vivo).

The antibody of the invention is not normally irradiated until after administration and distribution within the body, for example 2 hrs to 10 days after irradiation, for example between days 1 to 8, example on day 3 or 4.

Administration of the enzyme will generally take place at about the time irradiation, either shortly before, concurrently or shortly after, for example within 2 hours of irradiation. The pro-drug is not normally administered until after irradiation, for example 2 hrs to 7 days later, for example on day 1 or 4. The time delays following administration of the antibody and enzyme allow for clearance of undesired sequestered material from other parts of the body. The delays may be omitted but this is not presently considered to be preferred.

The areas to be irradiated will be where the tumour is located or where a site is at risk (such as an adjacent lymph node). Most suitably the tumour to be treated will be one readily illuminated because of its location, for example one adjacent to an area which can be reached by an appropriate source of light energy directly or via a light guide such an optical fibre. Thus tumours occurring in the lung, gastro-intestinal tract, urinary tract, testos, ovaries and body cavities, are particularly appropriate sites. Tumours in organs such as the lung, stomach, bowel and prostrate are readily irradiatable without needing to penetrate the body whereas other areas such as the liver and kidneys are accessible by minimally invasive surgical methods if visible or UV light (such as UV-A) is employed. Use of X-rays also allows opaque tissues to be treated.

Since the less toxic pro-drug is converted to active cytotoxic agent only in the locality of the tumour to be treated this invention enables the cancerous tissue to be treated with less toxic effects in remote tissues than would occur if an effective amount of cytotoxic drug were given.

From an alternative view, this invention provides a Fab or Fab¹₂ antibody fragment reversibly inhibited by a photocleavable moiety.

In this invention the photocleavable moiety may be located at or about the active site of the antibody. This may be achieved by incorporation of a single moiety at an active site for example by the methods of P Mendel et al., J. Am. Chem. Soc., 113, 275-2760 (1991).

In a preferred manner the photocleavable moiety may be incorporated into the antibody by coating the antibody with one or more usually more than one such moieties. Suitable methods include those of the publications by S Thompson et al referred to hereinbefore. Thus preferred antibodies (including fragments) of this invention are those which contain more than one photocleavable moiety, for example 2-25, more aptly 3-15, for example 4-8. If only one such moiety is included it is generally at the active site.

The bispecific antibodies of this invention may be prepared from individual antibodies, or preferably from the Fab or Fab₂¹ fragments of the individual antibodies by standard methods.

The antibodies of this invention which also comprise a ligand may be prepared by labelling the antibody with the ligand either before or after the photocleavable moiety is bound to the antibody.

In the instances where only binding site directed against the macromolecule is effected by coating, then the coating procedure may be performed on the antibody. (This may be where other reactive sites are absent or where they are protected, for example by absorption on an immunoabsorbant column). In the more common case where coating could effect, for example the other antibody component of the bi-specific antibody, then the coating is best performed on one component before coupling to form the bi-specific antibody.

The bifunctional antibody of this invention may then by prepared by linking the first antibody component and the reversibly inhibited second antibody component in a manner known in the art for preparing befunctional antibody. For example see the disclosures on bifunctional antibody preparation in “Monoclonal Antibodies”, Production, engineering in clinical applications, Postgraduate Medical Science, Edited by Mary, A. Ritter and Heather M. Ladyman, Cambridge University Press (1995) ISBN 0521473543; in particular the section by S Songsivilai and P J Lachmann at pages 121-135.

In an immunoassay where an antigen is captured onto a solid surface and then a second labelled antibody is added to detect the captured antigen, the antigen may be reacted with an anti-antigen antibody in solution as a primary capture reagent and then capture the complex on a solid surface by means of a standard secondary capture reagent (such as a second antibody) which can be employed for different primary capture antibodies and thus different tests against different analytes. A problem is that after the capture antibody is reacted with the antigen it must then be contacted with the surface coated with the secondary capture agent. This introduces a disturbance of the system either by the solution of analyte and antibodies being transferred or the solid phase being added. If, however, an antibody the form of this invention is employed as follows such disturbance is made unnecessary as the coated surface may be present during the initial reactions, secondary reaction with its activity being delayed until required and then switched on by exposure to e.g. UV light. Thus the antibodies of this invention are also of use in immunoassays

The location of specific biological cells is critical to both life systems and industrial processes. For example, bodily defence systems work by the localisation of specific cells such as T-cells, polymorphonuclear cells, macrophages, etc. to sites where foreign agents or aberrant cells are identified. A great deal of interest has been shown in developing means of increasing the attraction of cells to such sites, especially when the aberrant cells are tumour cells or infected cells such as cells infected with the immunodeficiency virus (Berg et al, Proceedings of the National Academy of Sciences of the United States of America, 88(11):4723-7,1991). Ligands, such as steroids and other substances which bind cellular membranes, receptors, enzymes, adhesion molecules, nucleic acids and antibodies are all classes of substances which can be used for such cell localisation.

Bispecific reagents, which have an affinity for both the site and the cell to be localised at the site may be made from these specific combining partners. Indeed, the specific combining abilities of specific antibodies against tumour cells on one side and the cells intended for localisation on the other side have already been used in this regard. Bispecific (and trispecific—Tutt et al, Journal of Immunology, 147(1):60-69, 1991) antibodies have been made for such localisation. These are composed of an antibody binding site against an antigen epitope of the tumour cell which, in the bispecific case, is connected to other antibody binding site against an antigenic epitope on the cell type (or virus—Mady et al, Journal of Immunology, 147(9):3139-3144, 1991) that is it is intended to localise at the tumour site. The specificity of the antibodies thus used is helpful in achieving localisation, however, it is important to minimise localisation of cells such as cytotoxic and inflammatory cells at sites other than those specifically targeted. A means of achieving greater localisation than obtainable by antibody alone would confer a distinct advantage on the approach. This has now been achieved as disclosed in this specification. This is done as follows:

A bispecific reagent is made having a binding site for the site to which localisation is to be directed and a second binding site for cells which are to be localised at the site, however, at least one of these binding sites is inhibited from being able to bind until activated Such inhibition may be achieved by means of coating the second binding site with a photolytically cleavable residue which inhibits the site from cell binding until photolytically cleaved. Thus reagents of the following forms may be employed in the manner of this invention:

(i) the reagent has its specific binding affinity for the site to which localisation is to be directed uninhibited whilst having its binding affinity for the cells to be localised reversibly inhibited;

(ii) the reagent has its specific binding affinity for the site to which localisation is to be directed inhibited and its specific binding affinity for the cells to be localised uninhibited;

(iii) the reagent has its specific binding affinity for the site to which localisation is to be directed and also its specific binding affinity for the cells to be localised reversibly inhibited.

An example of the use of such a localising reagent is as follows:

A bispecific antibody is made comprising an antibody site against a tumour cell and a reversibly inhibited antibody site against a cytotoxic cell such as a natural killer cell may and is given to an individual with a tumour site against which the bispecific antibody will bind.

It is allowed to localise at the site. In common with other bispecific antibodies other fractions will be taken up non-specifically in the reticuloendothelial system and specifically elsewhere such that it exists in spleen, liver, kidneys etc. The site to be treated is then exposed to light (such as UV-A light) causing activation of the anti-cell antibody binding site of bispecific antibodies bound at that location. These cells are then localised at that site and have their effect.

There are number of advantages of the approach. It is well known that antibody directed therapy is not as specific as desired (Bagshaw K D, page 79 of New Antibody Technology and the Emergence of Useful Cancer Therapy, Ed. Begent & Hamblin, published by the Royal Society of Medicine Press Ltd. 1995). The approach of this specification builds upon this specificity. At sites at which the bispecific antibody is not activated, the cells will not be specifically localised by the antibody, however, within the sites illuminated the antibodies will be activated. Importantly, not the whole site illuminated will necessarily thus contain an equal covering of activated antibody but it will be differentially present across the site in relation to the density of antigenic epitope found there. A limited degree of non-specific binding will be expected and antibody doing this within the illuminated area will be activated. However, the non-specific binding will be a small fraction of that had an uninhibited bispecific antibody been employed. Another advantage of the method is that with a conventional bispecific antibody, as soon as it enters the individual (by for example injection into a vein) it is able to bind to the cells to be localised. This was highlighted as a problem in the use of antibodies containing anti-CD3 specificity by Barr et al (Int. J. Cancer, 43, 501-597; 1991) stating that “Specific antibodies containing anti-CD3 specificity will not be suitable for systemic administration in humans since they will be absorbed by all peripheral lymphocytes”. This would clearly decrease the ability of the antibody to penetrate to the sites to be treated.

The localising cells of this specification includes viruses. As far as the localising cells are concerned they may be (i) cells already present within the individual or either normally or caused to increase before treatment by, for example, causing inflammation to occur at an area of the body or (ii) cells added from another immune or non-immune individual or previously taken from the individual himself and optionally treated (by culture to enhance their effectiveness, for example: lymphokine activated killer (LAK) cells (Nippon et al, Journal of the Nippon Medical School, 58 (6): 663-672); cloned cells such as T-cells may also be used. Additionally, cells may be employed as carriers of cytotoxic agents or substances, for example the encapsulation of recombinant human IFN gamma in human red blood cells targeted against adenocarcinoma (Chokri et al. Research in Immunology, 143 (1): 95-99, 1992). Similarly a radio-active isotope, a toxic such as diphtheria toxic, boron (for subsequent boron capture treatment) may be carried or the cell itself may be virally infected.

The antibodies specific for the foreign agents and cells to be localised may be obtained by standard means. In many instances it is preferable that they are of low inherent immunogenicity themselves. Humanised antibodies can, therefore, be used with good effect in bispecific antibody technology (Shalaby et al, Jorunal of Experimental Medicine, 175: 217-225, 1992). It is preferable that they be monoclonal antibodies but polyclonal antibodies may also be employed. Tumour cell targets include those described in the earlier patent application (ours—incorporated) some of which have already been used in targeting of bispecific antibodies (ovarian carcinoma—Segal et al, Immunobiology, 185; 390-402, 1992); breast cancer (Shi et al, Journal of Immunological Methods, 141, (2): 165-75); colon carcinoma (anti-CEA) (Jantscheff et al, Journal of Immunological Methods, 163 (1): 91-97, 1993); colon carcinoma (Beun et al, Journal of Immunological Methods 150 (6): 2305-2315, 1993); leukemic B-cells (Bohlen et al, Journal of Immunological Methods, 173 (1): 55-62, 1994); small cell lung cancer (Azuma & Niitani, Japanese Journal of Thoracic Disease, 29 (9): 1132-1137, 1991); adenocarcinoma (Chokri et al, Research in Immunology, 143 (1): 95-99, 1992); ovarian and breast carcinomas (Journal of Experimental Medicine, 175: 217-225, 1992).

Similarly, many different antibodies have been made against suitable cells to be localised, for example: T-cells (review) (Bolhuis et al, Journal of Cellular Biochemistry, 47: 306-310, 1991); cytotoxic T-cell clones (Haagen et al, Clinical and Experimental Immunology, 90 (3): 368-75, 1992); cytotoxic T-cells and activated peripheral blood lymphocytes (van Ravenswaay et al, Gynecological Oncology, 52 (2): 199-206, 1994); CD3+ lymphocytes (Malygin et al, Immunology, 81 (1), 92-95, 1994); CD16+ lymphocytes (Nitta et al, Immunology Letters, 28 (1): 31-37, 1991); Fc gamma R111, the low affinity Fc gamma receptor for polymorphonuclear leucocytes, macrophages and large granular lymphocytes (Garcia de Palazzo et al, International Journal of Biological Markers, 8 (4): 233-239, 1993); B-lymphocyte markers (Demanet et al, International Journal of Cancer—Supplement, 7: 67-68, 1992); myeloid cells (Ball, et al, Journal of Hematotherapy, 1 (1): 85-94, 1992), T Lymphocyte CD2, CD3, CD4, CD8 (Tutt et al. Journal of Immunology, 147 (1): 60-69, 1991); dengue virus (Mady et al. Journal of Immunology, 147 (9): 3139-3144, 1991 ), lymphokine activated killer (LAK) cells (Nippon et al. Journal of the Nippon Medical School, 58 (6): 663-672); NK cells or monocytes (Curnow et al, Scandinavian Journal of Immunology, 36 (2): 221-231, 1992).

So-called bispecific antibody “forks” have been described by (Ring et al, Cancer Immunol. Immunotherapy, 39 41-48; 1994). These are antibodies which have two specificies against two different markers on a tumour cell. For the fork to bind a tumour cell will require greater specificity than a single site would require and also the avidity of the antibody for the cell would be higher than with a single binding.

Employing the coating technology of the present specification it would be possible to coat one or both binding sites which might interfere with the localisation of the fork and only uncover it in the location of the tumour cell. That would reduce the chance of the antibody being removed by binding cells binding a single specificity on the way to the tumour cells.

Bispecific antibodies may be made in a number of ways, for example:

By chemically coupling an antibody of one specificity to another antibody of the other specificity by any of the wide variety of chemical means available for such coupling (such as glutaraldehyde, or preferably, one of the many more specific cross linkers such as SPDP-(N-Succininimidyl-3-[2-pyridyldithio]propionate). In this specification antibody means an intact antibody containing the usual number of binding sites and an Fc region or any fragment thereof which contains the antigen binding site (such as Fv, Fab, (Fab′). Suitable methods for the production of bispecific antibodies are described by H. Paulus, Behring Inst. Mitt., 78, 118-132, 1985. Small antibody fragments have particular advantages and may be prepared as described by Holliger et al, Proceedings of the National Academy of Sciences of the United States of America, 90 (14): 6444-6448; 1993).

In addition bispecific antibodies can be produced by bringing together two or more antibodies by means of cross-linking binders such as anti-antibodies, lectins or reagents such as protein A (Ghetie & Mota, Mol. Immunol. 17, 395-401: 1980).

The heady and light chains of the antibodies can be separated and allowed to recombine with each other, some chains thereby combining with chains of the other antibody forming bispecific antibodies (Paulus, Behring Inst. Mitt., 78, 118-132, 1985; Lebegue et al, C.R. Acad. Sci. Paris, Serie 111, 310, 377-382, 1990);

By the formation of quadromas from the two hybridomas synthesising the two antibodies from which a bispecific antibody is required (Suresh et al, Methods in Enzymology, 121, 211, 1986; Bos R, Nieuwenhuitzen W, Hybridoma, 11 (1): 41-51, 1992);

By genetic recombinant means such as described in COS-1 cells for the production of a bifunctional murine:human chimeric antibody (De Sutter & Fiers, Molecular Immunology, 31 (4): 261-267).

The bispecific reagent may have any specific binding partner as either one of its specificities.

The features required by a specific binding partner to be useful in the manner of this invention are as follows: that it can be coupled to another binding partner and still retain significant specific binding affinity for the other member of its binding pair; that it or a binding partner to which it is intended to be coupled may be reversibly inhibited from binding to the member of its own binding pair.

Classes of such specific binding partners are within, for example: antibodies, enzymes, ligands and receptors including biotin and avidin, adhesion molecules (such as ICAM-1, E-Selectin, VCAM-1, 1-SELECTIN and Endothelin-1), lectins, nucleic acids (such as DNA & RNA).

The coated reagent may have any number more than one of its sites reversibly inhibited. If one or more of the sites are to remain uninhibited this may be achieved by treating the sites to be inhibited before subsequently coupling them together with uninhibited sites or by taking advantage of differences in susceptibility to inhibition by different sites either as a result of their natural formation or by specifically reducing the likelihood that a site will be inhibited by adding the inhibitory substance in the presence of a specific combining partner for the site. For example, in the case where a bispecific reagent is required between and antibody of specificity K for a tumour cell marker and specificity L for a T-cell and in which the L specificity was required to be inhibited whilst the K was not, a bispecific antibody could be made by any of the art methods and that bispecific antibody bound with antigen K whilst the sites against L were inhibited. The reagent used for causing inhibition of the sites could then be withdrawn and the bispecific antibody separated from antigen K to provide the species required.

As well as therapeutic uses outlined above molecules of the form of this invention can have many utilities in allowing the remote localisation of cells in industrial processes such as in large scale cullular processes producing, for example, bio-products. A bispecific reagent can be initially added to the system, allowed to localise to a particular region and then when conditions are suitable, it can be activated, allowing the concentration of particular cells.

Many diagnostic applications of this technology are also envisaged. For example, in those instances where a particulate analyte, such as a cell-type, is to be measured it can be advantageous to allow the bispecific antibody to remain in dispersed in solution (where favourable fast reaction kinetics exist) while binding the cell and to then be subsequently activated such that it takes the cell to the location required, such as solid surface, for the analysis to be completed. Similarly in those instances where a particulate reagent is employed in a assay, it can be advantageous for sequential binding of this nature to be introduced. For example, to enable a single reagent mixture (comprising for example: a bispecific antibody with an activity for a particulate surface reversibly inhibited and another activity for an analyte; a particulate surface to which the bispecific antibody will bind when activated; a labelled antibody against the analyte) to be added to a sample, allowing the binding of the sample to proceed with the bispecific reagent still in solution (where reactions tend to be faster than on a solid surface) and then subsequently activating the binding activity for the particulate surface, after which the particulate surface is separated and the label determined.

An example of an assay of this form would be:

1. A bispecific antibody is made capable of binding an analyte A and a latex particle labelled with an antigenic epitope B but with its activity towards A reversibly inhibited;

2. A suspension of the latex particles, the bispecific reagent and a labelled antibody against the analyte in solution is contacted to analyte A allowing binding with the bispecific reagent in solution and the labelled antibody;

3. The inhibited activity of the bispecific antibody is activated allowing binding to the latex particles;

4. The latex particles are separated the label determined and related to analyte concentration.

It is particularly surprising that the efficiency of activation can be high enough to cause the binding of cells to their location sites (low efficiencies would be likely to produce insufficient attachment sites for cells).

The present invention provides an antibody which is capable of binding to a member of a binding pair and to a macromolecule; in which the capability of binding to the macromolecule is reversibly inhibited by the presence of the photocleavable moiety; wherein said macromolecule is present on a cell such that said antibody is capable of binding to said cell.

The member of a binding pair may also be a cell. Hence is a particularly suitable embodiment this invention provides an antibody which is capable of binding to a first cell and to a second cell, in which the capability of binding to the second cell is reversibly inhibited by the presence of a photocleavable moiety.

The first cell is normally one which it is desirable to eliminate or at least reduce, for example a tumour cell, cell of a parasite such as the malaria parasite or the like. Most desirably the first cell is a tumour cell, for example as hereinbefore named.

The second cell is normally one capable of disadvantaging the first cell when localised in the environs of the first cell. Although the term cell is to interpreted broadly also to include viruses, aptly the cell is a prokaryotic or eukaryotic cell and is most favourably a mammalian cell and is preferably a human cell. The most preferred second cells include cells of the human immune system which can kill undesired cells, for example those killer cells named hereinbefore.

It will be appreciated that a highly favoured member of a binding pair envisaged is a cell bound receptor, such as tumour markers so that the antibody binds to tumour cell possessing said marker.

In a preferred class the antibody of this invention has two active sites. This may be achieved in any convenient manner but it is present considered most appropriate to join two antibodies raised against two different epitopes. This type of antibody is often referred to as a bispecific antibody. Although normally this type of antibody has two specifities, more than two, for example three, is also envisaged but less preferred.

In a preferred aspect this invention provides a bispecific antibody comprising a first antibody component capable of binding a first cell bound receptor and a second antibody component capable of binding a second cell bound receptor; said second antibody component being reversibly inhibited by the presence of a photcleavable moiety.

Most aptly the first cell bound receptor is a receptor on a tumour cell (aptly a tumour marker). Most aptly the second cell bound receptor is a receptor on localising cell (aptly a killer cell as hereinbefore indicated).

The first antibody component may be the whole immunoglobulin (ie IgG) or more suitably a part thereof which retains the active site but is free of the Fc region, for example the Fab or Fab¹₂.

### EXAMPLE A

### The Use of a Reversibly Inhibited Bispecific Antibody to Specify the Location of Killing of Tumour Cells

The monoclonal antibody anti-CD-3 OKT3 (from the American type culture collection) against T-cells is taken and treated with 1-(2-nitrophenyl)ethanol (NPE) as follows:

First, 0.66 g 2 nitroacetoacetophenone is dissolved in a round bottomed flask in 7 ml of industrial methanol in the presence of NaBH4. The contents are swirled continuously to mix for 60 minutes. Ten ml H2O are then added to the flask with one drop 1M HCl. Ten ml of ethyl acetate is added and the mixture shaken thoroughly in a separating funnel. The bottom aqueous layer is run off and discarded. The washings are repeated with another 10 ml H2O. The top layer is collected and placed in an evaporating flask with MgSO4 to remove all traces of water. The preparation is passed through filter paper and the clear NPE eluate collected. The methanol is then removed on a rotary evaporator at 60C leaving a small quantity of viscous material. This is left overnight open in a fume cupboard. 31.3 μl of di-phosgene is added to a solution of 44 mg NPE and 20.6 μl of pyridine in 1 ml of dry dioxan. A white precipitate immediately forms and the reaction is allowed to go to completion over 15 minutes. The reaction mixture is then evaporated in a stream of nitrogen for 45 minutes to remove unreacted material and the off-white nitrobenzyloxycarbonyl chloride (NPE-COCl) is resuspended in 1 ml of dioxan. The following steps in this example are then carried out with protection of the light-sensitive reagents to light. One hundred μl of this was then added to 15 ml of the anti-CD-3 OKT3 antibody dialysed at a concentration of 0.5 mg/ml against 0.1M NaHCO3 at pH8.3 and gently rotated for 4hr. The solution was then dialysed against 0.9% NaCl. Antibody retaining binding to T-cells is then removed by exposing the treated antibody to a suspension of T-cells at 4C, incubating removing the supernatant solution and then purifying it by HPLC chromatography. The antibody is then conjugated with a monoclonal antibody against human colon carcinoma cells LoVo by means of standard treatment with a heterobifunctional cross linker such as (N-succinimidyl-3-[2-pyridydithio]proprionate) (SPDP) and further purified to obtain a purified preparation of 1:1 conjugates by HPLC.

Following the approach by Barr (Int. J. Cancer, 43, 501-507; 1991) the cytotoxicity of the bispecific antibody against 51Cr-labelled LoVo cells is then assessed in microtitre plates by means of the standard cytotoxicity assay of Brunner et al (In: B. Bloom & J. R. David (eds), In vitro methods in cell-mediated and tumour-immunity, p.423, Academic Press, New York (1976). Those wells which receive cytotoxic T-cells, labelled LoVo cells, NPE-inhibited bispecific antibody and are irradiated with UV-A light by exposure to a Spectroline EN-16/F UV lamp (Spectronics Corporation, Westbury, N.Y.) for 15 minutes show greater cytotoxicity than the same mixture without irradiation or the mixture without the NPE-inhibited antibody but with 15 minute irradiation. Enhanced cytotoxicity is also observed over the controls if the bispecific antibody is first exposed to the TV-A source for 15 minutes while held in quartz-glass cuvettes before addition to the cytotoxicity test wells.

### EXAMPLE B

### The Use of a Reversibly Inhibited Bispecific Antibody in the Rapid Determination of Low Levels of Micro-Organisms in a Water Sample

Two monoclonal antibodies (P & Q) are obtained by standard means against the micro-organism E.coli which are shown to be able to both bind the micro-organism simultaneously. This is done by standard means which involve briefly: (1) coating a microtitre plate with monoclonal antibody P (2) adding a suspension of the micro-organism, incubating this and then washing off unbound material and (3) adding the monoclonal antibody Q [which had been suitably labelled with alkaline phosphatase by means of the Pierce & Warriner (UK) Maleimide Alkaline Phosphatase Conjugation Kit (1995 cat number 31492)] allowing it to bind, washing off unbound material and (4) determining the alkaline phosphatase remaining. A substantial enzyme reactivity compared to other immunoassays employing this plate format, proves that the two antibodies can bind the micro-organism simultaneously.

The following part of this example are then carried out with protection of the light-sensitive reagents from light. A Monoclonal antibody R is then obtained against bovine serum albumin (BSA) and then conjugated with NPE as in Example 1 of this specification. Antibody still able to react with BSA is removed by means of exposure to an immunoabsorbent, BSA-beaded agarose (Sigma Chemical Co. Ltd., 1995 cat. no. A3790). The remaining conjugate is then coupled to monoclonal antibody P employing SPDP and purifying the 1:1 conjugates by means of HPLC, again as in Example 1 of this specification. This conjugate Z is then used in the test as follows:

Polypropylene cuvettes of thickness 1 cm, 2 cm width and 5 cm high are taken and 1 ml of a 1% solution of BSA added into their bottoms in a coating buffer of 50 mM bicarbonate pH 9.3 and left for five minutes for coating to take place. The solutions are then withdrawn by means of a pastuer pipette and the cuvettes washed six times with a washing solution of 50 mM tris pH 7.4 plus 0.02% Tween 20). 0.2 ml of a 20 μg/ml solution of conjugate Z are then placed in the cuvettes followed by eight ml of the water samples to be tested and standards containing known numbers of E.coli organisms, with mixing. The mixtures are then incubated for five minutes at room temperature to allow binding between E.coli present and conjugate Z. After this time the broad face of the cuvettes are exposed to UV-A light of the same intensity as above for a further fifteen minutes with gentle agitation. Further light protection is then unnecessary. The solutions are removed and the cuvettes washed six times with washing solution. 2 ml of a 10 mM solution of substrate para-nitrophenol phosphate in 50 mM bicarbonate buffer pH 10.3 containing 3.3 MgCl2 are then added and the cuvettes incubated until a suitable change at their absorption at 405 nm had taken place for the cuvette with the highest standard. A graph is plotted of absorption change against E.coli amount for the standards and the unknown samples read off against this. The results are superior than those obtained from a parallel example in which uninhibited bispecific antibody is added for in this case some bispecific antibody combines with the BSA in the cuvette before the E.coli in contrast to the case with the inhibited bispecific antibody where it remains in solution for a sufficient time for the E.coli binding to take place in the favourable solution environment.

### EXAMPLE 1

Inhibited Antibody Against Alkaline Phosphatase and CEA

a) A polyclonal antibody against alkaline phosphatase (Sigma Chem. Co. Ltd., 1994 catalogue no. p5521) by standard procedures and finally purified by means of an alkaline phosphatase affinity chromatography column.

b) 1-(2-nitrophenyl)ethanol was treated with di-phosgene in dioxane to yield nitrophenethyloxycarbonyl chloride (S Thompson et ad, Biochem. Biphys. Res. Comm., 201, 1213-1219 (1994).

c) A monoclonal antibody against tumour antigen CEA was obtained in standard manner from a hybridoma cell line.

d) Aliquots of nitrophenylethyloxycarbonyl chloride were added to 1 mg of purified anti-alkaline phosphatase in 1 ml of 0.1M NaHCO₃ solution. The resulting material was dialysed against 0.9% sodium chloride solution.

e) The monoclonal antibody against tumour antigen CEA was conjugated to the reversibly inhibited anti-akaline phosphatase antibody by means of the heterobifunctional cross-linker 3-(2-pyridyldithio)propionic acid N-Hydroxysuccinimide ester (SPDP-Sigma Chemical Co. Ltd. 1994 cat. no. P3415) as follows: the anti-alkaline phosphatase antibody (2 mg, 12 mg/ml) and the monoclonal antibody(4.6 ml, 5.2 mg/ml) were each dialysed against 0.1M potassium phosphate, 0.1 M NaCl, pH 7.5 (coupling buffer) and incubated separately for 2 h at room temperature with eightfold molar excess of SPDP (125 μul of a 3.2 mg/ml solution of SPDP in ethanol was added to each sample). The monoclonal antibody was redialysed against coupling buffer and the anti-alkaline phosphatase was dialysed against 0.1M sodium acetate, 0.1M Na Cl pH 4.5. Dithiothreitol was then added to the anti-alkaline phosphatase to a final concentration of 0.02M. After 30 minutes at room temperature, the anti-alkaline phosphatase was passed through a Pharmacia PD10 column equilibrated with coupling buffer, and immediately added to the monoclonal antibody. After 4 hr incubation at room temperature 1 mg of iodoacetamide was added and the protein was eluted on a 2.5×90 cm Ultrogel AcA 22 column in borate buffered saline, pH 8.5. Polymerised material was eluted in two fractions and concentrated using a Millipore CX-100 immersible membrane.

### EXAMPLE 2

Reduction in Viability of Tumour Cells

A cell line obtained from a human carcinoma was grown and aliquots placed in two microtitre plates at a density of 10,000 cells per well in incorplete modified Dulbecco's medium with 10% foetal calf serum (IMDM). The wells of both plates then received 200 μl of a 5 μg/ml solution of cAb in IMDM apart from one row which received only IMDM (the ‘zero control’). The plates were then incubated for one hour at 4° C. and unbound antibody removed by three change of the culture medium. On plate was then exposed to IV-A light in a similar means to that described in S Thompson et al, Biochem. Biophys. Res. Com., 201, 1213-1219 (1994). The pro-drug etoposide phosphate was synthesised as described in Senter, P D., Saulner M G., Schrelber G J., Hirschberg D L., Brown J P., Helstrom K E, Proc. Natl. Acad. Sci. USA, 85, 4842-4846 (1988) from the parent compound etoposide (Sigma Chemical Co. Ltd. 1994 cat no. E 1383) was added over a ranze of concentrations to groups of wells and incubated at 37° C. for 6 hours, the cells are then washed a further 12 hours. The cells in the invididual wells are then assessed for their viability and it is seen that the cells in the wells previously illuminated by the UV-A light were not viable. That this was an effect due to the binding of alkaline phosphatase in the illuminated wells and consequently a greater activation of the pro-drug in those wells was demonstrated by the cells in the zero control remaining viable after exposure to the UV-A.

### EXAMPLE 3

Example 1 was repeated making the bispecific (F(ab′)2 conjugate from F(ab′)2 fragments of both the antibodies following the method described in Glennie M J, Brennand D M, Bryden F, Stirpe F, Woth A T & Stephenson G T (1987), Preparation and performance of bispecific F(ab′γ)2 antibody containing thloether-lined Fab′γ fragments, Journal of Immunology, 139, 2367-2375.

### EXAMPLE 4

The procedure of Example 1 was repeated using the linker M2C2H of the Pierce and Warriner 1995 catalogue (cat. No. 22304) instead of SPDP.

### EXAMPLE 5

The procedure of Example 3 was repeated using the linker M2C2H of the Pierce and Warriner 1995 catalogue (cat. No. 22304) (ie 4-(N-maleimidomethyl)-cyclohexane-1-carboxylhydrazide) instead of SPDP.

### EXAMPLE 1A

Association of Alkaline Phosphatase on Colon Carcinoma Cells by Means of a Bispecific Antibody the Enzyme Binding Activity of which was Modulatable by Ultraviolet Light Irradiation

Preparation of the Bispecific Antibody Conjugate

Monoclonal antibodies against Carcinoembrionic antigen (CEA) and alkaline phosphatase (AP) (Zymed Laboratories Inc. clone number ZAP1 cat. no. 03-2200) were obtained.

The monoclonal anti-AP was first coated with NPE. The antibody, at a concentration of 0.5 mg/ml, was first dialysed overnight against 0.1M NaHCO3. During dialysis the volume increased from 3 to 4 ml. 1 m was retained as a control and two 1.5 ml fractions taken. Nitrobenzylcarbonyl chloride was prepared by treating NPE with di-phosgene in dioxan [Senter, P D, Tansey, M J, Lambert, J M & Blattler, W A (1985), Photochem. Photobiol., 42, 231-237] and 10 μl of this was added to one 1.5 ml fraction and 20 μl was added to the other. The samples were allowed to react with gentle rotation for 4 hr and were then dialysed against 0.9M NaCl. The final concentration of the control sample was 0.21 mg/ml and of the treated samples 0.135 and 0.105 mg/ml. The samples were found to have been coupled to around the same extent (averaging 8 and 12 NPE residues per antibody molecule) and were consequently mixed. The coated antibody was used without further purification. Both the NPE-coated anti-AP antibody and the anti-CEA antibody at 0.18 ml/m were derivatised with SPDP: 1 ml aliquots of both antibodies were dialysed at a concentration of 0.1 ml/ml against 0.1M phosphate buffer pH 7.5 containing 0.1M NaCl. 25 μl of SPDP (0.63 ml/ml in ethanol) was then added to each antibody. The antibodies were then left for 30 minutes to react. The anti-AP was dialysed against 0.1M phosphate buffer pH 7.5 and the anti-CEA antibody and left for 30 minutes at room temperature. The preparation was dialysed against two batches of 0.1M phosphate buffer pH 7.5. The anti-CEA antibody was then reduced and added to the NPE-anti-AP antibody and left to react for 24 hours at 20° C. The conjugate was then dialysed against distilled water to remove unwanted reaction products. The yield was shown by electrophoresis to be ca. 80% with at least 75% of the antibodies conjugated.

Targeting of AP to CEA-Coated ELISA Plates

Nunc microtitre plates were coated with 10 μg/ml CEA (Calbiochem 1994/5 cat. no. 219368). The bispecific antibody conjugate was then either irradiated or not for 6 minutes with UV light and 50 μl of each at 10 μg/ml were diluted to 1 ml in Tris buffer pH 7.4 containing 0.05% Tween and doubling dilutions added to wells across the plate. Both native anti-CEA and anti-AP antibodies were similarly treated as controls. After two hours the plate was washed and 100 μl AP at 2 μg/ml or, in control wells, an anti-mouse IgG labelled with alkaline phosphatase (at 1:1000 dilution) was added in Tris-Tween buffer. The AP (Biogenesis cat. no. 0300-1004) was added to directly detect the presence of active conjugate which had bound to the CEA, the anti-IgG(AP) as a positive control to confirm the SPDP coated anti-CEA antibody (either conjugated or not) could still bind to the CEA on the plate. The AP was visualised in all wells by the addition of nitro-phenolphosphate substrate under standard conditions and the reaction monitored spectrophotometrically at 405 nm. This showed that irradiating the bispecific antibody resulted in more AP being bound the microtitre plate wells than was bound with non-irradiated bispecific antibody.

Targeting of AP to Colon Cancer Cells Expressing CEA

CEA producing colon cancer cells were grown in microtitre plates to approximately 90% confluence, this time in 200 μl DMEM medium per well containing 10% foetal calf serum. 100 μl of the medium was then removed and replaced with free serum free medium for 1 hr to accustom the cells to serum free media (SFM). All media was then removed and 100 μl of SFM containing the bispecific antibody conjugates at 2.5 μg/ml which had either not been irradiated or after irradiation by 2 ml of the bispecific antibody conjugate sample being irradiated in saline for 6 minites in a quartz cuvette at a distance of 0.5 cm from a spectroline EN/16/F UV lamp (Spectronics Corporation, Westbury, N.Y.) with an emission peak of 365 nm. The assay was performed with 10 replicate wells in each case. After 2hrs all of the media was again removed and 100 μl of SFM containing AP (2 μg/ml) was added. After a final 2hr incubation the cells were extensively washed (five times) with buffered saline and 100 μl of substrate buffer containing AP substrate pNPP was added and the AP activity then monitored at 405 nm with a microtitre plate reader. Controls were added in which no bispecific conjugate was added and also in which bispecific conjugate had been added but no AP. This provided a measurement of the natural levels of AP in the cells and also allowed us to prove that the cells to which AP had been added had been washed sufficiently to remove all non-specifically absorbed AP.

The results showed that the wells containing unirradiated bispecific conjugate only showed a background level of AP which was the same as wells not receiving the bispecific antibody or added AP. In contrast, the wells which had received the irradiated bispecific antibody conjugate demonstrated much higher levels of AP activity as a result of this being bound to the CEA producing cells throught the active bispecific antibody conjugate.

### EXAMPLE 2A

Irradiation in Culture Medium

Example 1A (last section) was repeated with an enzyme amplified system employing an AMPAK kit according to the manufacturer's instructions for the detection of AP in microtitre plate assays (Dako Diagnostics Ltd. U.K.) which produced a readily identifiable red colour in place of the para-nitrophenolphosphate to investigating any possible effect of culture medium during the irradiation of bispecific antibody conjugate. In this the conjugate, at 40 μg/ml was irradiated in quartz cuvettes in 0.9% saline or SFM for 6 minutes. The antibodies were then diluted to 5 μg/ml in either SFM or media contianing 10% FCS before being added to 10-fold replicate wells contianinl the CEA-producing colon cancer cells. Table 1 shows the visually scored results of this from which it was clear that much more AP was bound by the wells receiving irradiated bispecific antibody conjugate, irrespective of whether the irradiation had been carried out in SFM or saline, than those which had received the unirradiated bispecific antibody conjugate.

TABLE 1
The visual results were scored from 0 to a maximum of 10.

| | |
|---|---|
| Plate Blank | 1 |
| No irradiation | 3 |
| Irradiation in saline | 10 |
| Irradiation in SFM | 10 |

### EXAMPLE 3A

Time of Irradiation

Example 1A (last section) was repeated but here, the enzyme amplified system was used as in Example 2A and also the unirradiated bispecific antibody conjugate at concentrations of 2.5 μg/ml and 5.0 μg/ml was added directly to all of the wells of the plate after which the cells were irradiated from below the plate for times of: 0, 3, 6, 9 and 12 minutes. As shown by the results in Table 2, even with the attenuation of the irradiation by the plastic plate, increasing AP capture was strongly related to increasing time of irradiation.

TABLE 2
The results were scored from 0 to a maximum of 10 as follows:

| | conjugate Visual | conjugate Visual |
|---|---|---|
| Irradiation | Colour 2.5 μg/ml | Colour 5.0 μg/ml |
| Time (min) | development | development |
| 0 | 1 | 4 |
| 3 | 3 | 5 |
| 6 | 7 | 8 |
| 9 | 8 | 10 |
| 12 | 10 | 10 |

### EXAMPLE 3B

Phosphorylated etoposide is obtained as described by Haisma et al in Cancer Immunol. Immunother. (1992) 34:343-348. Example 2A is repeated but instead of the enzyme amplified system being added etoposide phosphate is added as described in Haisma et al. Tumour cell death is greater in those cultures not exposed to the irradiated bispecific conjugate than to the unirradiated bispecific conjugate.

The CEA producing cells are grown in flasks and accustomed to serum-free medium as in Example 1A. They are then plated in microtitre plates and a solution of 100 μg/ml in serum-free culture fluid added to half the wells. The other half receives the same bispecific antibody preparation but which has been irradiated through 2 ml of the NPE-coated bispecific antibody sample being irradiated in serum-free culture fluid for 6 minutes in a quartz cuvette at a distance of 0.5 cm from the Spectroline lamp. The cells are incubated for 1 hr at 37° C. after which they are washed by gentle centrifugation and change of medium. 100 μ SFM containing alkaline phosphatase at 2 μg/ml is added to each well and inculated for 30 minutes at 37° C. after which they are washed by gentle centrifugation and a solution of 100 μl epoposide phosphate (10 μM), made according to Senter et al Proc. Natl. Acad. Sci. USA (1988) 85: 4842, in SFM added to each well and incubated at 37° C. for 3hr. The cells are again washed and grown for three days whereupon their viability is assessed by the sulphorhodamine-B assay. It is found that those wells having received irradiated bispecific antibody show lower cell viability than those receiving unirradiated antibody.

In-situ Irradiation

The CEA producing cells are grown in flasks and accustomed to serum-free medium as in Example 1A. They are then plated in microtitre plates and a solution of 100 μl NPE-coated bispecific antibody made as in Example 1 at a concentration of 10 μg/ml in serum-free culture fluid is added to the wells. Half of the wells are irradiated as described in Example 3A. The cells are incubated for 1 hr at 37° C. after which they are washed by gentle centrifugation and change of medium. 100 μl SFM containing alkaline phosphatase at 2 μg/ml is added to each well and incubated for 30 minutes at 37° C. after which they are washed again and a solution of 100 μl epoposide phosphate (10 μM), made according to Senter et al Proc. Natl. Acad. Sci. USA (1988) 85: 4842, in SFM added to each well and incubated at 37° C. for 3hr. The cells are again washed and grown for three days whereupon their viability is assessed. Control wells are also set up which do not receive either the NPE-bispecific antibody, alkaline phosphatase or eptoposide phosphate or combinations of the three. When the cell viability is assessed by the sulphorhodamine-B assay as described by Skehan et al (1990). New calorimetric cytotoxicity assay for anticancer-drug screening, J. Natl. Cancer Inst. 82: 1107 those wells having been given the NPE-bispecific antibody, alkaline phosphatase and etoposide phosphate and then exposed to the UV light show greater cell death than those in unirradiated wells or in control wells irradiated or not.

### EXAMPLE 4A

### Use of a Bispecific Antibody having a Reversibly Inhibited Binding Site for alkaline phosphatase and Binding Affinity for Carcinoembrionic Antigen to Locate alkaline-phosphatase-bound Particulate Beads with Cells Bearing Carcinoembrionic Antigen after the Bispecific Antibody had been Uninhibited by Irradiation with Ultraviolet Light

Preparation of the Bispecific Antibody Conjugate

Monoclonal antibodies against Carcinoembrionic antigen (CEA) and alkaline phosphatase (AP)(Zymed Laboratories Inc. clone number ZAP1 cat. no. 03-2200) were obtained.

The monoclonal anti-AP was first coated with NPE. The antibody, at a concentration of 0.5 mg/ml, was first dialysed overnight against 0.1M NaHNO3. During dialysis the volume increased from 3 to 4 ml. 1 ml was retained as a control and two 1.5 ml fractions taken. Nitrobenzylcarbonyl chloride was prepared by treating NPE with di-phosgene in dioxan [Senter, P D, Tansey, M J, Lambert, J M & Blattler, W A (1985), Photochem. Photobiol., 42. 231-237] and 10 μl of this was added to one 1.5 ml fraction and 20 μl was added to the other. The samples were allowed to react with gentle rotation for 4hr and were then dialysed against 0.9M NaCl. The final concentration of the control sample was 0.21 mg/ml and of the treated samples 0.135 and 0.105 mg/ml. The samples were found to have been coupled to around the same extent (averaging 8 and 12 NPE residues per antibodymolecule) and were consequently mixed. The coated antibody was used without further purification. Both the NPE-coated anti-AP antibody and the anti-CEA antibody at 0.18 mg/ml were derivatised with SPDP: 1 ml aliquots of both antibodies were dialysed at a concentration of 0.1 mg/ml against 0.1M phosphate buffer pH 7.5 containing 0.1M NaCl. 24 μl of SPDP (0.63 mg/ml in ethanol) was then added to each antibody. The antibodies were then left for 30 minutes to react. The anti-AP was dialysed against 0.1M phosphate buffer pH 7.5 and the anti-CEA was dialysed against 0.1M sodium acetate pH 4.5 both being dialysed overnight at 4° C. After dialysis 100 μl of 0.5M DL-dithiothreitol (DTT) was added to the anti-CEA antibody and left for 30 minutes at room temperature. The preparation was dialysed against two batches of 0.1M phosphate buffer pH 7.5. The anti-CEA antibody was then reduced and added to the NPE-anti-AP antibody and left to react for 24 hours at 20° C. The conjugate was then dialysed against distilled water to remove unwanted reaction products. The yield was shown by electrophoresis to be ca. 85% with at least 75% of the antibodies conjugated.

Preparation of the Beads

Carboxylate modified intensely blue coloured polystyrene beads of three different sizes (0.22 mm, 0.4 mm and 0.8 mm) were obtained. The beads were coupled with alkaline phosphatase (AP) by first washing them three times by resuspending 100 ml of 10% beads in 1 ml of distilled water and pelleted by centrifugation at 10000 RPM for 10 minutes. The beads were then resuspended in 120 ml of MES buffer in 770 ml distilled water with 230 ml of N-hydroxysuccinimide at 50 mg/ml in distilled water (NHS), 1-Ethyl-3-(3-dimethylaminopropyl)carbodiimide hydrochloride (EDAC) at 50 mM/ml was prepared and 100 μl immediately added to the bead preparation, the beads were then left for 2 hours and washed once with 0.1 MES buffer pH 4.7 and then with 0.1M bicarbonate pH 8.4. 500 μl of AP at 2 mg/ml was then added to the pelleted beads. The volume was adjusted to 1 ml in the same buffer and left to react overnight. The next day the beads were washed three times, centrifuged in microcentrifugation-tubes and then resuspended in 1 ml phosphate buffered saline.

The protein concentration was calculated for each set of beads. 80% of added AP was found to have bound to each set.

Capture of AP-Labelled Beads by Activated Bispecific Antibody Conjugate on Cells

CEA producing colon cancer cells were grown in microtitre plates to approximately 90% confluence, this time in 200 μ DMEM medium per well containing 10% foetal calf serum. 100 μ of the medium was then removed and replaced with free serum free medium for 1 hr to accustom the cells to serum free media (SFM). All media was then removed and 100 μl of SFM containing the bispecific antibody conjugates at 2.5 μg/ml which had either not been irradiated or after irradiation by 2 ml of the bispecific antibody conjugate sample being irradiated in saline for 6 minutes in a quartz cuvette at a distance of 0.5 cm from a spectroline EN/16/F UV lamp (Spectronics Corporation, Westbury, N.Y.) with an emission peak of 365 nm. The assay was performed with 10 replicate wells in each case. After 2hr all of the media was again removed and 10 μl of each bead suspension followed by 90 μl of serum free medium added to their wells and left for 24hr. The plate was emptied, washed 5 times with phosphate buffered saline and 100 μl of 5 mM para-nitrophenol phosphate in 50 mM bicarbonate buffer pH 10.3 containing 3.3 mM MgCl₂ buffer was added and the enzyme product measured at 405 nm with a microtitre plate reader. This was confirmed by measurement of the alkaline phosphatase activity on the beads where with all three bead sizes more activity was found associated with those wells which had received irradiated conjugate than those which had not.

### EXAMPLE 5A

An anti-CD3 (T lymphocyte activating marker) monoclonal antibody is coated with NPE and coupled to the anti-CEA monoclonal antibody was was the anti-alkaline phosphatase antibody in Example 1A. This is then divided into two aliquots, one being irradiated as in Example 1A and the other untreated. They are then compared in their ability to target T-cells obtained and cause lysis of the human cancer cells as described in Barr et al, Int. J. Cancer (1989), 43, 501-507 but employing the concentrations of antibody conjugate described in Example 1A above (Targeting of AP to colon cancer cells expressing CEA). More cell death is obtained with the irradiated fraction of the bispecific antibody than that not irradiated.

In Situ Targeting

The NPE-coated anti-CD3-anti-CEA bispecific antibody conjugate is employed in situ activation as follows: They are then compared in their ability to target T-cells obtained and cause lysis of the human cencer cells as described in Barr et al. The CEA-bearing cells are plated and accustomed to serum free medium as described in Example 1A above (Targeting of AP to colon cancer cells expressing CEA). Antibody conjugate is added in serum-free medium (SFM) at a concentration of 2.5 μg/ml and incubated at 37° C. for two hours, other wells being control wells and not receiving antibody conjugate. Wells are irradiated as described in Example 3A and the cells are washed in SFM. The ability of added T-cells to lyse the CEA-expressing cells is then assessed following Barr et al. More cell lysis is obtained in irradiated wells than those not irradiated or those control wells not receiving the bispecific antibody, whether or not irradiated.

### Lists of Receptors and their Ligands

The receptor may be:

neurotransmitter receptors

gastric receptors such as enterogastrone, cholecystokinin, pancreozymin and secretin

histamine receptors, serotonin receptors

alpha and beta-adrenoceptors

aldrenalin receptors, noradrenalin receptors

enkephalis receptors, endorphin receptors

melanine stimulating hormone receptor

acetylcholine receptor

other homone receptors such as insulin receptors and growth hormone receptors

thyroxin, thyrotropin, glucagon, progesterone, oestradiol, testosterone, folcile stimulating hormone, luteinizing hormone, chorionic gonadotropin, placental lactogen, oxytocin, vasopressin, erythropoietin, renin and angiotenin, ACTH receptors corticosterone, cortisol, 5α-dihydrotestosterone, aldosterone, 1.25-dihydroxycholecalciferol, parathyroid hormone and calcinonin receptors

corticorophin releasing factor receptors

cytokine receptors such as Tumour Necrosis Factor Receptor

Immune receptors such as Histocompatability antigen receptors, T-cell receptors and B-cell receptors

specific drug receptors such as diethylstilbestrol (oesradiol-17β receptor)

specific carbohydrate structures

enzymes may be used as receptors

synthetic receptors are being increasingly made

The ligand may be

gastric hormones and factors such as:

enterogastrone, cholecystokinin, parcreozymin, histamin and secretin neurotransmitters such as: serotonin, adrenalin, noradrenalin, enkephalins, endorphins, melanine stimulating hormone, acetylcholine, insulin receptors and growth hormone, thyroxin, thyrotropin, glucagon, progesterone, oestradiol, testosterone, folcile stimulating hormone, luteinizing hormone, chorionic gonadotrophin, placental lactogen, oxytocin, vasopressin, erythropoietin, renin and angiotensin, ACTH, corticosterone, cortisol, 5α-dihydrotestosterone, aldosterone, 1,25-dihydroxycholecalciferol, parathyroid hormone and calcitonin, corticorophin releasing factor, cytokines such as Tumour Necrosis Factor, Immune agents such as Histocompatability antigens, T-cell factors and B-cell factors and antigens, specific drugs such as diethylstilbestrol (oesradiol-17β receptor) enzyme substrates, especially irreversible inhibitors

ligands for synthetic receptors

specific carbohydrate binding agents such as: Wheat-germ agglutin, Pokeweed

mitrogen (may be ligands or receptor)

1. An antibody which is capable of binding to (a) a member of a binding pair and (b) a macromolecule in which the capability of binding to the macromolecule is reversibly inhibited by the presence of a photocleavable moiety.

2. Antibody as claimed in claim 1 which is a bispecific antibody comprising a first antibody component capable of binding a receptor and a second antibody component capable of binding a macromolecule.

3. An antibody as claimed in claim 2 wherein the first and second antibody components are parts of antibodies which retain the active site but are free of the Fe regions.

4. An antibody as claimed in claims 1-3 wherein the first antibody component is against a tumour cell marker.

5. An antibody as claimed in any of claims 2-4 wherein the second antibody components is against an enzyme.

6. An antibody as claimed in claim 5 wherein the enzyme is capable of converting a pro-drug of a cytotoxic drug into the cytotoxic drug.

7. An antibody as claimed in any of claims 1-6 wherein the photocleavable moiety is 1-nitrophenylethan-1-ol conjugated to the antibody.

8. A method of treating a tumour which comprises administering an antibody as claimed in any of claims 4-7 exposing the tumour to electromagnetic energy, optionally administering an enzyme if desired, and administering a non-cytotoxic pro-drug.

9. An antibody as claimed in any of claims 1-4 and 7 where the macromolecule is on a cell.
